SEQUENCE LISTING (12 sequences)

SEQ ID NO: 1 - protein, 211 aa, Artificial Sequence
  Domain 3 of truncated Gal-3BP
LLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQS

SEQ ID NO: 2 - protein, 125 aa, Artificial Sequence
  Domain 4 of truncated Gal-3BP
FQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 3 - protein, 336 aa, Artificial Sequence
  Domains 3-4 of truncated Gal-3BP
LLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 4 - protein, 591 aa, Artificial Sequence
  tGal-3BP-Fcfusion protein
MYRMQLLSCIALSLALVTNSISLLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVDTAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1764 bp, Artificial Sequence
  Nucleic acid of tGal-3BP-Fc fusion protein
atgtacaggatgcaactcctgtcttgcattgcactaagtcttgcacttgtcacgaattcgatatccctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggacagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 6 - DNA, 66 bp, Artificial Sequence
  Nucleic acid of secretary signal peptide of tGal-3BP-Fc fusion protein
atgtacaggatgcaactcctgtcttgcattgcactaagtcttgcacttgtcacgaattcgatatcc

SEQ ID NO: 7 - DNA, 1008 bp, Artificial Sequence
  Nucleic acid of domains 3-4 of tGal-3BP-Fc fusion protein
ctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggac

SEQ ID NO: 8 - DNA, 690 bp, Artificial Sequence
  Nucleic acid of IgG Fc fragment of tGal-3BP- Fcfusion protein
agatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 9 - DNA, 19 bp, Artificial Sequence
  G3BP-F858
ttccacaagctggcctctg

SEQ ID NO: 10 - DNA, 18 bp, Artificial Sequence
  G3BP-R2004
aggaggggagcctgcagt

SEQ ID NO: 11 - DNA, 26 bp, Artificial Sequence
  EcoRV-G3BP-F927
aggatatcnctcctcccccaggaccc

SEQ ID NO: 12 - DNA, 27 bp, Artificial Sequence
  G3BP-R1934-BglII
agagatatgtccacacctgaggagttg

(19) United States
(12) Patent Application Publication
(10) Pub. No.: US 2016/0347835 A1
(43) Pub. Date: Dec. 1, 2016

(54) METHOD OF INHIBITING METASTASIS OF CANCER CELLS AND MODULATING AUTOIMMUNE DISEASES USING GAL-3BP-Fc FUSION PROTEIN

(71) Applicant: Show-Chwan Memorial Hospital, Changhua (TW)

(72) Inventors: KUEN-DER YANG, Taichung City (TW); CHI-CHEN WEI, Changhua City (TW); ENG-YEN HUANG, Kaohsiung City (TW); KENG-LIANG WU, Kaohsiung City (TW); YA-CHU CHUANG, Kaohsiung City (TW)

(21) Appl. No.: 14/724,282

(22) Filed: May 28, 2015

(51) Int. Cl. C07K 16/28; C07K 14/00

(52) U.S. Cl. CPC C07K 16/283; C07K 14/00; C07K 2319/30; C07K 2319/33; C07K 2317/76

(57) ABSTRACT

A tGal-3BP-Fc fusion protein, a pharmaceutical composition, an isolated nucleic acid, a recombinant expression vector, and a method of inhibiting, decreasing, reducing, suppressing or limiting metastasis of cancer cells, and immunosuppressing or modulating phagocytosis and T-cell functions by using the same are disclosed herein. The tGal-3BP-Fc fusion protein comprises: a truncated Gal-3 binding protein with domain 4, and at least an Fc fragment of an immunoglobulin G. A pharmaceutical composition comprises the tGal-3BP-Fc or Fc-tGal-3BP fusion protein, and a pharmaceutically acceptable carrier. The method comprises a step of administrating the pharmaceutical composition to a subject in a therapeutically effective amount to inhibit, decrease, reduce, suppress or limit invasiveness and metastasis of cancer cells or to treat or modulate immune reactions of inflammatory diseases in the subject.

### BACKGROUND OF THE INVENTION

### 1. Field of the Invention

The present invention generally relates to a fusion protein, a pharmaceutical composition, an isolated nucleic acid, a recombinant expression vector, and a method of inhibiting, decreasing, reducing, suppressing or limiting metastasis of cancer cells, and modulation of inflammation by using the same.

### 2. Description of the Related Art

Current research indicates that galectins (Gal) play important roles in diverse physiological and pathological processes, including immune and inflammatory responses, tumor development and progression, neural degeneration, atherosclerosis, diabetes, and wound repair.

Thus, galectins may be a therapeutic target or employed as therapeutic agents for inflammatory diseases, cancers and several other diseases.

Given its known biological roles in tumor progression, Gal-1 has been postulated as an attractive target for anticancer therapies. In particular, there is evidence that Gal-1 produced by tumor contributes to tumor immune escape; thus Gal-1 inhibitors could be used to counter this effect and enhance anti-tumor immunity. A vast amount of literature also suggests that Gal-3 plays an important role in tumor progression and metastasis, by functioning at a number of different points (Liu, et al. 2005). Importantly, even though Gal-3 has been implicated in diverse biological pathways associated with tumor development and progression, in almost all cases, the effect of Gal-3 is cancer-promoting. Thus, Gal-3 inhibitors may be useful for treatment of various cancers by suppressing various pathways.

At present, cancer target therapy is usually directed against growth factor receptors and/or its signal transduction pathways to induce cancer cell apoptosis or to suppress cancer cell invasiveness. However, tumor growth and metastasis are determined not only by cancer invasiveness, but also escape of anti-tumor immunity.

Recently, specific cancer target therapy by monoclonal antibodies or small molecules that suppress cancer cell migration and proliferation by blocking growth factor receptors and/or its signal transduction pathways has raised a hope for the control of cancer cell invasiveness. Most of the specific cancer target therapies are focused on single target and directly against cancer cells, but much less on augmentation of anti-tumor immunity or on both suppression of cancer invasiveness and enhancement of anti-tumor immunity.

Galectins, such as Gal-1 or Gal-3, are known to suppress or augment immune functions (Liu, et al. 2012). Moreover, inventors have also recently found that Gal-1 is involved in radio resistance of cancer cells and inhibition of Gal-1 improved the radio resistance (Huang, et al. 2012). Competition or scavenge of galectins may decrease cancer cell invasiveness, and modulate immunity and autoimmunity. Gal-3 and its binding protein called 90K have been implicated in pathogenesis of rheumatoid arthritis (Ohshima, et al. 2003; Li, et al. 2013).

### REFERENCE

1. Liu F T and Rabinovich G A. Galectins as modulators of tumor progression. Nat Rev Cancer. 5:29-41, 2005
2. Liu F T, Yang R Y, Hsu D K. Galectins in acute and chronic inflammation. Ann NY Acad Sci. 1253:80-91, 2012
3. Huang E Y, Chen Y F, Chen Y M, Lin I H, Wang C C, Su W H, Chuang P C, Yang K D. A novel radioresistant mechanism of galectin-1 mediated by H-Ras-dependent pathways in cervical cancer cells. Cell Death Dis. 3:e251, 2012
4. Ohshima S, Kuchen S, Seemayer C A, Kyburz D, Hirt A, Klinzing S, Michel B A, Gay R E, Liu F T, Gay S, Neidhart M. Galectin 3 and its binding protein in rheumatoid arthritis. Arthritis Rheum. 48(10):2788-95, 2003.
5. Li S, Yu Y, Koehn C D, Zhang Z, Su K. Galectins in the Pathogenesis of Rheumatoid Arthritis. J Clin Cell Immunol. 30;4(5), 2013.

### SUMMARY OF THE INVENTION

Therefore, it is a primary objective of the present invention to provide a fusion protein comprising truncated Gal-3 binding protein (Gal-3BP) and at least an Fc fragment of an immunoglobulin, called Gal-3BP-Fc, a pharmaceutical composition comprising the same, an isolated nucleic acid and a recombinant expression vector encoding the same, and a method of inhibiting, decreasing, reducing, suppressing or limiting metastasis of cancer cells, and modulation of immune functions such as phagocytosis and cell immunity by using the same.

To achieve the foregoing objective, the present invention provides a fusion protein, comprising: a truncated Gal-3 binding protein with domain 4; and at least an Fc fragment of an immunoglobulin G (IgG).

Preferably, the truncated Gal-3 binding protein may further comprise at least a fragment of domain 3 of Gal-3 binding protein.

Preferably, the fusion protein may further comprise a signal peptide linked to an N-terminus of the truncated Gal-3 binding protein.

Preferably, the fusion protein may have an Fc fragment linked to the C-terminus or N-terminus of the truncated Gal-3 binding protein.

Preferably, the Fc fragment may comprise binding affinities against Fc receptors (FcRs) on cells with an FcR.

Preferably, the truncated Gal-3 binding protein may comprise N-glycosylation for binding of Gal-1, Gal-3 or other galectins.

Preferably, the truncated Gal-3 binding protein may consist of a sequence of SEQ ID NO: 2.

Preferably, the truncated Gal-3 binding protein may consist of a sequence of SEQ ID NO: 3.

Preferably, the fusion protein may consist of a sequence of SEQ ID NO: 4.

Preferably, the immunoglobulin G (IgG) may comprise human IgG1 or IgG3.

To achieve the foregoing objective, the present invention further provides a pharmaceutical composition for inhibiting, decreasing, reducing, suppressing or limiting metastasis of cancer cells, and activating phagocytosis or/and T cell immunity, comprising: the fusion protein mentioned above; and a pharmaceutically acceptable carrier for inhibiting, decreasing, reducing, suppressing or limiting metastasis of cancer cells, and activating phagocytosis and/or T cell immunity.

Preferably, the fusion protein is present in a therapeutically effective amount to inhibit, decrease, reduce, suppress or limit metastasis of the cancer cells.

Preferably, the cancer cells overexpress galectins such as galectin-1, galectin-3 or a combination thereof on cell surface, in extracellular spaces or in blood circulation.

Preferably, the cancer cells are selected from a group consisting of colon cancer, rectal cancer, breast cancer, cervical cancer, gastric cancer, pancreatic cancer, prostate cancer, renal cancer, melanoma, and ovarian cancer.

To achieve the foregoing objective, the present invention further provides an isolated nucleic acid encoding the above-mentioned fusion protein.

To achieve the foregoing objective, the present invention further provides a recombinant expression vector comprising the isolated nucleic acid.

To achieve the foregoing objective, the present invention further provides a cell transformed or transfected with the recombinant expression vector.

Preferably, the cell may comprise eukaryotic cells, and the eukaryotic cells may comprise mammalian cells.

To achieve the foregoing objective, the present invention further provides a method of inhibiting, decreasing, reducing, suppressing or limiting metastasis of cancer cells, and enhancing or modulating anti-tumor immunity and anti-autoimmunity, comprising: administrating the pharmaceutical composition mentioned above to a subject in a therapeutically effective amount to inhibit, decrease, reduce, suppress or limit metastasis of cancer cells in the subject.

Preferably, the subject may be a mammal, and the pharmaceutical composition may be administered orally, intramuscularly, intraperitoneally, or subcutaneously.

To achieve the foregoing objective, the present invention further provides a method of modulation of T-cell functions, comprising: administrating the pharmaceutical composition mentioned above to a subject in a therapeutically effective amount, to compete with the cell surface glycoprotein receptors of T cell for galectins such as galectin-1, galectin-3 or a combination to reduce galectin-1-mediated or galectin-3-mediated T cell apoptosis.

Preferably, the subject may be a mammal, and the pharmaceutical composition may be administered orally, intravenously, intramuscularly, intraperitoneally, inhaled or subcutaneously.

To achieve the foregoing objective, the present invention further provides a method of modulating immune functions, comprising: administrating a pharmaceutical composition comprising the fusion protein mentioned above and a pharmaceutically acceptable carrier to a subject in a therapeutically effective amount, to compete with galectin-3 and/or galectin-3 binding protein (90K), and resulting in modulation of proinflammatory reactions or adaptive T cell polarization.

Preferably, the subject is a mammal.

Preferably, the proinflammatory reactions or the adaptive T cell polarization is relative to a disease comprising rheumatoid arthritis, autoimmune diseases, inflammatory diseases or macrophage activation syndromes.

The fusion protein, the pharmaceutical composition comprising the same, the nucleic acid sequences and the recombinant expression vector encoding the same, and a method of inhibiting, decreasing, reducing, suppressing or limiting metastasis of cancer cells, and enhancing or modulating anti-tumor immunity and anti-autoimmunity by using the same, so that the present invention has the following advantages:

(1) The fusion protein may interact, compete, or scavenge the overexpressed Gal-1, Gal-3 and/or other galectins on the cell surface, in the extracellular spaces or in blood circulation to inhibit, decrease, reduce, suppress or limit migration and metastasis of cancer cells, and enhancing or modulating anti-tumor immunity and anti-autoimmunity.

(2) The fusion protein with Fc fragment may attract white blood cells, such as phagocytes or granulocytes, and enhance phagocytosis or activate immune response towards the cancer cells with overexpressed Gal-1, Gal-3 and/or other galectins while inhibiting, decreasing, reducing, suppressing or limiting migration and metastasis of cancer cells.

(3) The fusion protein may bind, compete or scavenge circulating Gal-1, Gal-3 or other galectins to modulate immunity against infections, tumors or autoimmunity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The properties and effects of the present invention will now be described in more details hereinafter with reference to the accompanying drawings that show various embodiments of the invention as follows.

FIG. 1 is a schematic representation of the composition of the tGal-3BP-Fc fusion protein in accordance with an embodiment of the present invention, showing the Fc fragment is linked to C-terminus or N-terminus of the different length of tGal-3BP. The fusion protein comprises a truncated Gal-3 binding protein (Gal-3BP) with domain 4 and further comprises at least a fragment of domain 3; and at least a portion of an Fc fragment.

FIG. 2A is a schematic view of tGal-3BP-Fc modulation of phagocytosis and T-cell functions.

FIG. 2B shows tGal-3BP-Fc effectively scavenge Gal-3 levels; FIG. 2C shows an inhibiting effect of tGal-3BP-Fc on the Gal-3 mediated T-cell apoptosis; and FIG. 2D shows the effect of tGal-3BP-Fc on the Gal-3 mediated modulation of T helper cell type 1 polarization, showing a reduced expression of T-bet transcription factor.

FIG. 3A shows 8 representative stable clones of tGal-3BP-Fc-expressing CHO cells.

FIGS. 3B-3C show the expression of tGal-3BP-Fc protein in condition medium of tGal-3BP-Fc-expressing CHO cells measured by EIA method (FIG. 3B) and Western blot (FIG. 3C), respectively.

FIGS. 3D-3F show the expression of tGal-3BP-Fc protein in cytosol of tGal-3BP-Fc-expressing CHO cells measured by flow cytometric assay (FIGS. 3D and 3E) and Western blot (FIG. 3F), respectively.

FIGS. 4A-4B show the binding of tGal-3BP-Fc with Gal-1 or Gal-3.

FIG. 5 shows the binding of tGal-3BP-Fc and Fc receptor (FcR).

FIG. 6 shows the phagocytosis of tGal-3BP-Fc fusion protein in PMA-treated THP-1 macrophages.

FIG. 7 shows the inhibitory effects of tGal-3BP-Fc on binding of Gal-3 to mIgE.

FIG. 8 shows that tGal-3BP-Fc fusion protein inhibits Gal-3-induced migration of Gal-3-knockdown DLD-1 cells.

### DETAILED DESCRIPTION OF THE PREFERRED EMBODIMENTS

The technical content of the present invention will become apparent by the detailed description of the following embodiments and the illustration of related drawings as follows. Embodiments of the present invention will be explained in detail below with regard to the specific preferred embodiment. It should be noted, however, that further fusion proteins without departing from the scope and spirit of the invention set forth in the claims the invention may be manufactured by analogous means.

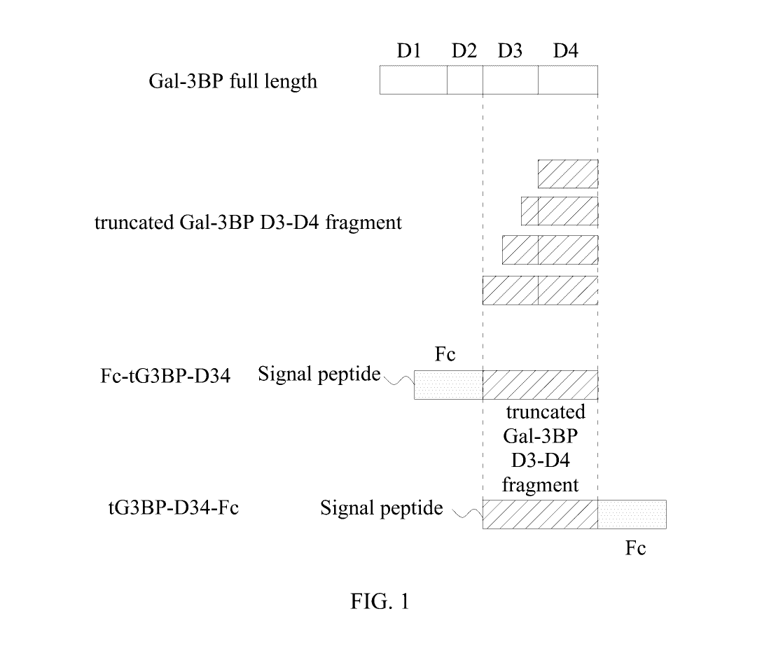

Please referring to FIG. 1, FIG. 1 is a schematic representation of the composition of the truncated Gal-3BP-Fc fusion protein (tGal-3BP-Fc). According to an embodiment of the present invention, the fusion protein comprises a truncated Gal-3 binding protein (Gal-3BP) with domain 4 and further comprises at least a fragment of domain 3; and at least a portion of an Fc fragment, a constant region of heavy chain in an immunoglobulin G.

In an embodiment, the truncated Gal-3BP may comprise the domain 4 and further comprises the fragment having at least a glycosylation side, located at C-terminus of Gal-3BP, directly fused with an Fc fragment, for example, a human IgG1 or IgG3 Fc fragment. That is, the tGal-3BP-Fc fusion protein is composed of one signal sequence fused to a truncated Gal-3BP between Leu250 and Asp585, at the N-terminus, followed by an Fc fragment with and without a linker fused to the C-terminus thereof. In another embodiment, the Fc fragment with and without a linker, followed by a signal sequence, may be linked to the N-terminus of the tGal-3BP which does not affect the efficacy or efficiency of the fusion protein. Preferably, the fusion proteins invented contain (indicated by slant line) the amino acid sequence of the domain 4 of the human Gal-3BP shown as SEQ ID NO: 2. Preferably, the fusion proteins invented contain (indicated by slant line) the amino acid sequence of the domains 3-4 of the human Gal-3BP shown as SEQ ID NO: 3. In a preferred embodiment, the fusion protein may comprise the amino acid sequences of signal sequence, domains 3-4 of human Gal-3BP, and the human IgG1 Fc fragment, as shown in SEQ ID NO: 4. In addition, the position of Fc fragment in the fusion proteins can be fused in N-terminus or C-terminus of the truncated Gal-3BP.

The Fc fragment of the fusion protein may comprise at least a portion of a constant region of an immunoglobulin, e.g. a constant region of a heavy chain or a light chain in the immunoglobulin. Preferably, at least a portion of the Fc fragment comprises at least a portion of a constant region of a heavy chain in the immunoglobulin G. The constant region of the heavy chain is preferably an Fc fragment comprising the CH2 and CH3 domain and, optionally, at least a part of the hinge region. The immunoglobulin may be an IgG, IgM, IgD or IgE immunoglobulin or a modified immunoglobulin domain derived therefrom. The IgG immunoglobulin may be selected from IgG1, IgG2, IgG3 or IgG4 domains or from modified domains such as described in U.S. Pat. No. 5,925,734. The immunoglobulin may exhibit effector functions, such as effector functions selected from antibody-dependent cellular cytotoxicity (ADCC) and/or complement-dependent cytotoxicity (CDC). In some embodiments, however, modified immunoglobulins having modified, e.g. at least partially deleted, effector functions may be used.

The fusion protein according to one embodiment of the invention may comprise an N-terminal signal sequence, which allows secretion from a host cell after recombinant expression. The signal sequence may be a signal sequence which is homologous to the truncated Gal-3BP of the fusion protein. Alternatively, the signal sequence may be a heterologous signal sequence, e.g. the Igκ or the Igλ signal peptide sequence as well. In a different embodiment, the fusion protein is free from an N-terminal sequence, thus representing the mature form of the fusion protein. In a preferred embodiment, the signal sequence may comprise signal sequence of IL-2, for example. The signal sequence of IL-2 will be cleaved between 22nd and 23rd a.a. of the tGal-3BP-Fc fusion protein after secretion.

In an embodiment, the Fc fragment effector in the tGal-3BP-Fc fusion protein may be functioned as effector to activate phagocytosis and ADCC through interactions with Fc receptors (FcγRs). Generally, different IgG isoforms exert different levels of effector functions. Particularly, human IgG1 and IgG3 have strong ADCC effects while human IgG2 and IgG4 exerts weak ADCC effects. In ADCC, the Fc region of an antibody binds to Fc receptors (FcγRs) on the surface of immune effector cells, i.e. WBCs, such as natural killer cells, macrophages, neutrophils, and mast cells leading to the phagocytosis or lysis of the targeted cells. Modifying effector functions can be achieved by engineering the Fc region to improve binding of FcγRs.

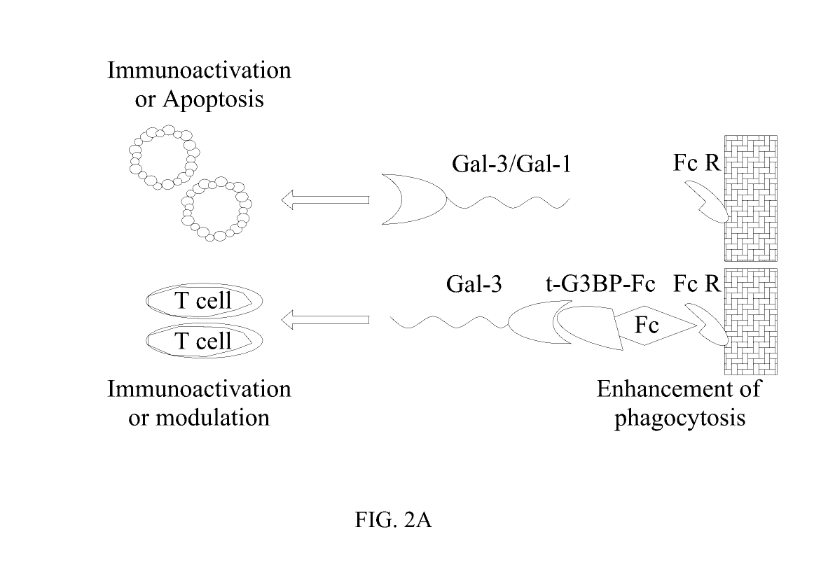

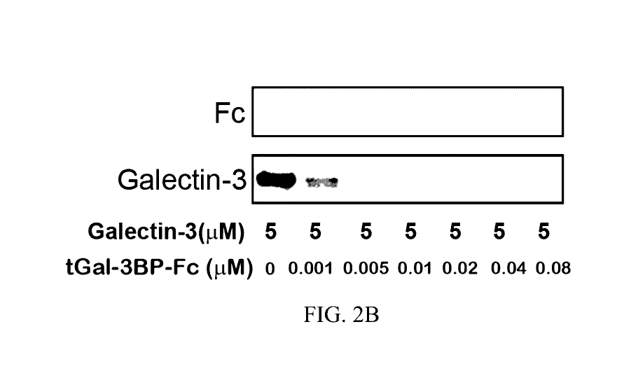

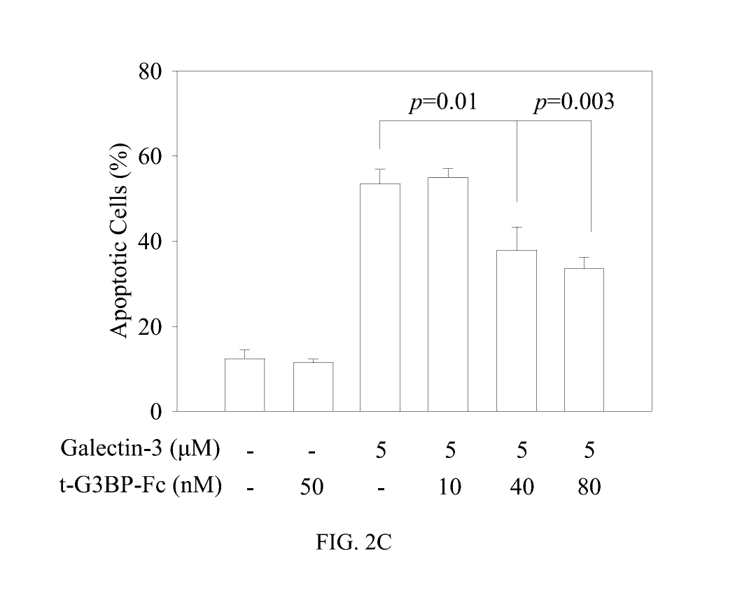

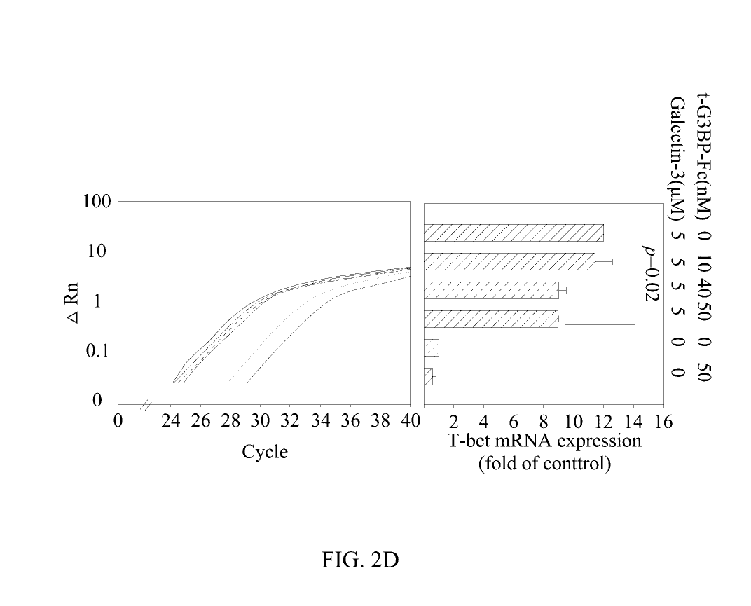

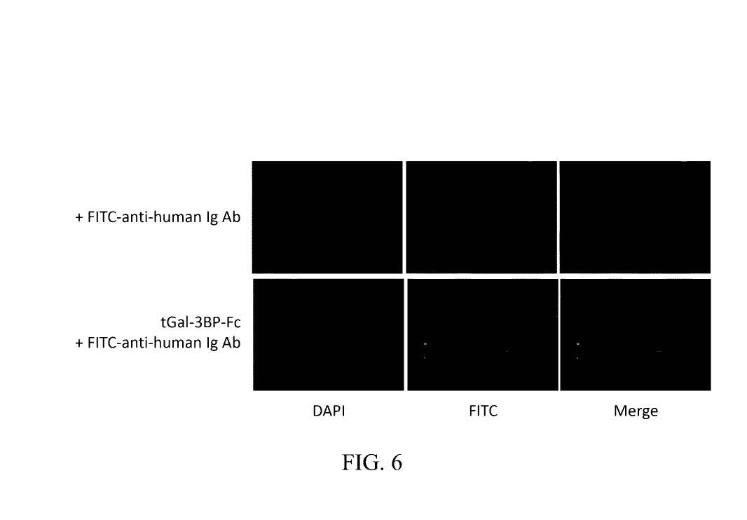

FIG. 2A is a schematic view of Gal-3 modulation of phagocytosis and T-cell functions. In circulation, there's Gal-3 that may interact with T-cell, leading to apoptosis of the T-cells. In an embodiment, tGal-3BP-Fc fusion protein may be added to interact with the Gal-3 while the Fc region of the tGal-3BP-Fc fusion protein binds to the Fc receptors (FcR) located on cell surface for phagocytosis by cells, including phagocytes, with an FcR. In other words, the Gal-3 bound with tGal-3BP-Fc may be phagocytosed and scavenged, and the Gal-3 may be reduced, at least a portion, such that the apoptosis and polarization of the T-cells may be modified. As shown in FIG. 6, addition of tGal-3BP-Fc, in contrast to the addition of Fc fragment, enhanced FITC-labeled Gal-3 phagocytosis by THP-1 macrophages. Moreover, scavenge of Gal-3 with tGal-3BP-Fc reduced the Gal-3 mediated T cell apoptosis (FIG. 2C), and significantly modulated the transcription factor T-bet expression of Th1 polarization (FIG. 2D).

The binging affinity of full length Gal-3BP binding with Gal-3 at the C-terminal of Gal-3BP is activated by glycosylation at the C-terminal of Gal-3BP. That is, the glycosylation of Asn113 and Asn149 of the domain 3 in Gal-3BP and Asn92 and Asn121 of the domain 4 in Gal-3BP promote the Gal-3BP binding with Gal-3. The N-terminal of Gal-3BP promotes cell-aggregating activity when Gal-3BP binding with Gal-3. However, high accumulation of Gal-3 at Gal-3BP may also induce Gal-3-meditated activation when the N-terminal of Gal-3BP is present, and may transfer when Gal-3BP is secreted form cell surface. The fusion protein of the present invention has removed the N-terminal of Gal-3BP to reduce side effect and the C-terminal of Gal-3BP is maintained to binding with galectins. In an embodiment, the truncated Gal-3BP comprises a domain 4 of Gal-3BP, which has two glycosylation sides. In another embodiment, the truncated Gal-3BP further comprises a fragment having at least a glycosylation side of domain 3 of Gal-3BP, but not limited to.

A further embodiment of the present invention relates to an isolated nucleic acid molecule encoding tGal-3BP-Fc fusion protein as described above. The nucleic acid molecule may be a DNA molecule, e.g. a double-stranded or single-stranded DNA molecule, or an RNA molecule. The nucleic acid molecule may encode the tGal-3BP-Fc fusion protein or a precursor thereof, e.g. a pro- or pre-proform of the tGal-3BP-Fc fusion protein which may comprise a signal sequence or other heterologous amino acid portions for secretion or purification which are preferably located at the N- and/or C-terminus of the tGal-3BP-Fc fusion protein. In a preferred embodiment, the isolated nucleic acid molecule encoding tGal-3BP-Fc fusion protein (shown in SEQ ID NO: 5) may consist of a secretary signal peptide (shown in SEQ ID NO: 6), DNA sequences between domains 3 and 4 of the Gal-3-BP (shown in SEQ ID NO:7), and a sequence of IgG Fc fragment (shown in SEQ ID NO: 8).

In one embodiment, the isolated nucleic acid molecule may be operatively linked to an expression control sequence, e.g. an expression control sequence which allows expression of the nucleic acid molecule in a desired host cell. The isolated nucleic acid molecule may be inserted to a vector, e.g. a plasmid, a bacteriophage, a viral vector, a chromosal integration vector, etc. Examples of suitable expression control sequences and vectors are described for example by Sambrook et al. (1989) Molecular Cloning, A Laboratory Manual, Cold Spring Harbor Press, and Ausubel et al. (1989), Current Protocols in Molecular Biology, John Wiley & Sons.

Various expression vector/host cell systems may be used to express the nucleic acid sequences encoding the tGal-3BP-Fc fusion proteins according to embodiments of the present invention. Suitable host cells include, but are not limited to, prokaryotic cells such as bacteria, e.g. E. coli, eukaryotic host cells such as yeast cells, insect cells, plant cells or animal cells, preferably mammalian cells. Further, embodiments of the present invention relate to a non-human organism transformed or transfected with a nucleic acid molecule as described above. Such transgenic organisms may be generated by known methods of genetic transfer including homologous recombination.

In an embodiment of the invention, a pharmaceutical composition for inhibiting, decreasing, reducing, suppressing or limiting migration or metastasis of cancer cells is disclosed. The pharmaceutical composition may comprise: the tGal-3BP-Fc fusion proteins mentioned above, and a pharmaceutically acceptable carrier for inhibiting, decreasing, reducing, suppressing or limiting migration or metastasis of cancer cells, and modulating phagocytosis or T cell functions. In an embodiment of the invention, a method of inhibiting, decreasing, reducing, suppressing or limiting metastasis of cancer cells, and modulating phagocytosis or T cell functions are disclosed. The method may comprise: administrating the pharmaceutical composition mentioned above to a subject in a therapeutically effective amount to inhibit, decrease, reduce, suppress or limit metastasis of cancer cells, and modulating phagocytosis or T cell functions in the subject. The pharmaceutical composition may be used in the treatment of cancers with overexpression of Gal-1, Gal-3 and/or other galectins, e.g. colon cancer, rectal cancer, breast cancer, cervical cancer, gastric cancer, pancreatic cancer, prostate cancer, renal cancer, melanoma, or ovarian cancer. The pharmaceutical composition may be also used in the treatment of inflammatory disorders with overexpression of Gal-1, Gal-3 and/or other galectins. Preferably, the pharmaceutical composition may be administered orally, intramuscularly, intraperitoneally, intravenously, inhaled or subcutaneously, to the subject.

Gal-1 or/and Gal-3 overexpression in a tumor or the tissue surrounding a tumor (stroma) were considered as a sign of the tumor's malignant progression and, consequently, of a poor prognosis for patients. Overexpression of Gal-3 and its binding protein (90K) have been demonstrated in patients with rheumatoid arthritis. As compared to normal tissues, the expression of Gal-1, Gal-3 and/or other galectins was increased in rectal cancer, breast cancer, cervical cancer, gastric cancer, pancreatic cancer, prostate cancer, renal cancer, melanoma, or ovarian cancer. Similarly, Gal-3 and 90K increased in patients with rheumatoid arthritis or osteoarthritis. Thus, tGal-3BP-Fc fusion protein may interact with overexpressed Gal-1 and Gal-3 on the cell surface, in the extracellular spaces and in blood circulation to inhibit, decrease, reduce, suppress or limit migration and metastasis of cancer cells, and to modulate phagocytosis or T cell functions of inflammatory disorders including autoimmune disorders. Moreover, the tGal-3BP-Fc fusion protein may further interact with white blood cells, such as phagocytes or granulocytes, and enhance or modulate immune response towards the cancer cells or inflammatory disorders with overexpressed Gal-1, Gal-3 or/and its binding protein while inhibiting, decreasing, reducing, suppressing or limiting migration and metastasis of cancer cells, and modulating phagocytosis or T cell functions.

Material and Methods

Cloning and Constructing of tGal-3BP-Fc Fusion Protein

Total RNA from polymorphonuclear cells (PMNs) was isolated and reverse transcribed into cDNA. The domains 3 and 4 of Gal-3BP were obtained by nested P CR with specific primers listed below in Table 1 (first-round P CR, G3BP-F858/G3BP-R2004; second-round P CR, EcoRV-G3BP-F927/G3BP-R1934-BglII). Different lengths of the tGal-3BP will be obtained from the P CR reactions with specific primers within the domains 3-4 of the Gal-3BP.

TABLE 1
A pair of specific primers for amplifying
tGal-3BP with domains 3 and 4.

| SEQ ID NO: | Primer Name | Sequences |
|---|---|---|
| 9 | G3BP-F858 | 5′-ttccacaagctggcctctg-3′ |
| 10 | G3BP-R2004 | 5′-aggaggggagcctgcagt-3′ |
| 11 | EcoRV-G3BP-F927 | 5′-agGATATC*ctcctcccccaggaccc-3′ |
| | | (*: A, T, C or G) |
| 12 | G3BP-R1934-BglII | 5′-agAGATATgtccacacctgaggagttg-3′ |

The amplified different P CR fragments code between Leu250 and Asp585 of the domains 3-4 in Gal-3BP (truncated Gal-3BP, tGal-3BP) were cloned into the EcoRV and BglII restriction sites of the pFUSE-hIgG1-Fc2 (InvivoGen, San Diego, Calif.) vector, designed for the construction of Fc-fusion proteins: tGal-3BP-Fc or Fc-tGal-3BP. Different lengths of the truncated Gal-3BP including at least a glycosylation side between domains 3 and 4 are also subcloned into the vector with Fc sequence at 5′ end and 3′ end of the fusion clones (FIG. 1). The recombinant proteins (tGal-3BP-Fc), which consists of a sequence of SEQ ID NO: 3, were expressed in CHO cells as fusion proteins with Fc region of an immunoglobulin G at the C-terminus or N-terminus. Zeoc was used to select the stable clones for tGal-3BP-Fc expression. The recombinant protein was purified using protein-A affinity chromatography. Fc fragment by itself was purified and used as a control for tGal-3BP-Fc in the embodiments. Medium from tGal-3BP-Fc-expressing CHO cells were harvested and coated on 96-well ELISA plates followed by colorimetrically detection by using streptavidin conjugated anti-human IgAb (1:2000) and TMB substrate. tGal-3BP-Fc-expressing CHO cells were permeabilized and detected with FITC conjugated anti-human IgAb (1:100 dilution) by using flow cytometer. Both condition medium and cell lysate of tGal-3BP-Fc-expressing CHO cells were detected by Western blot with streptavidin conjugated anti-hIgGAb (1:2000 dilution) and TMB substrate.

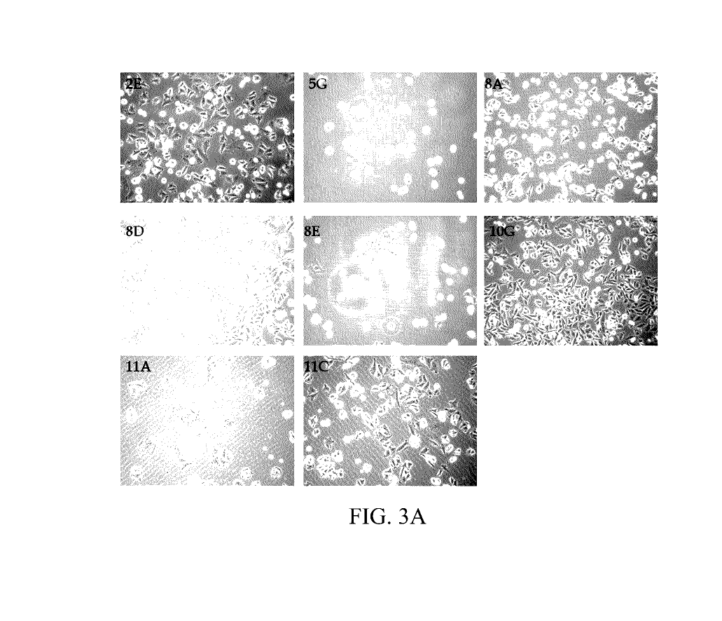

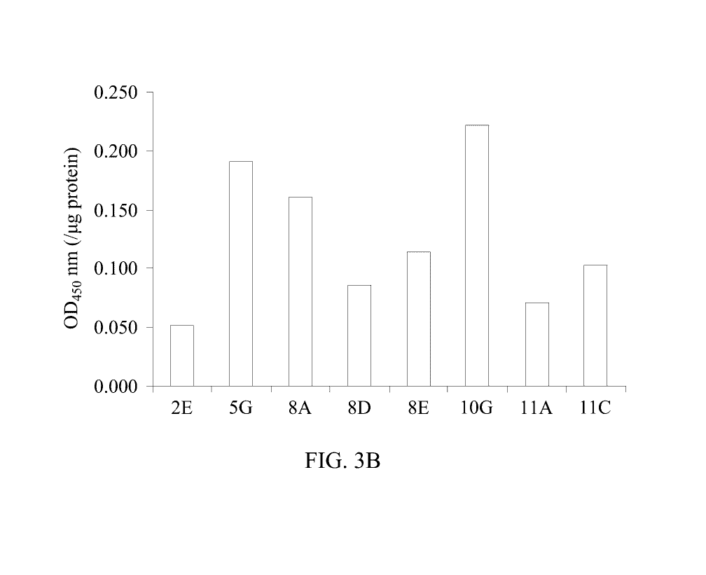

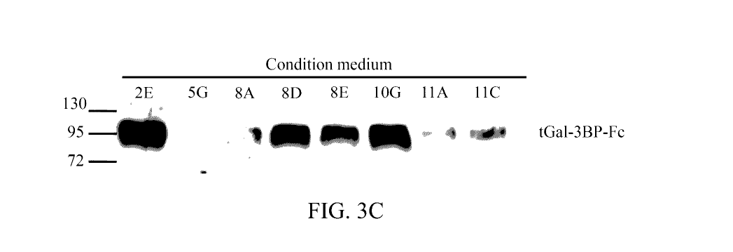

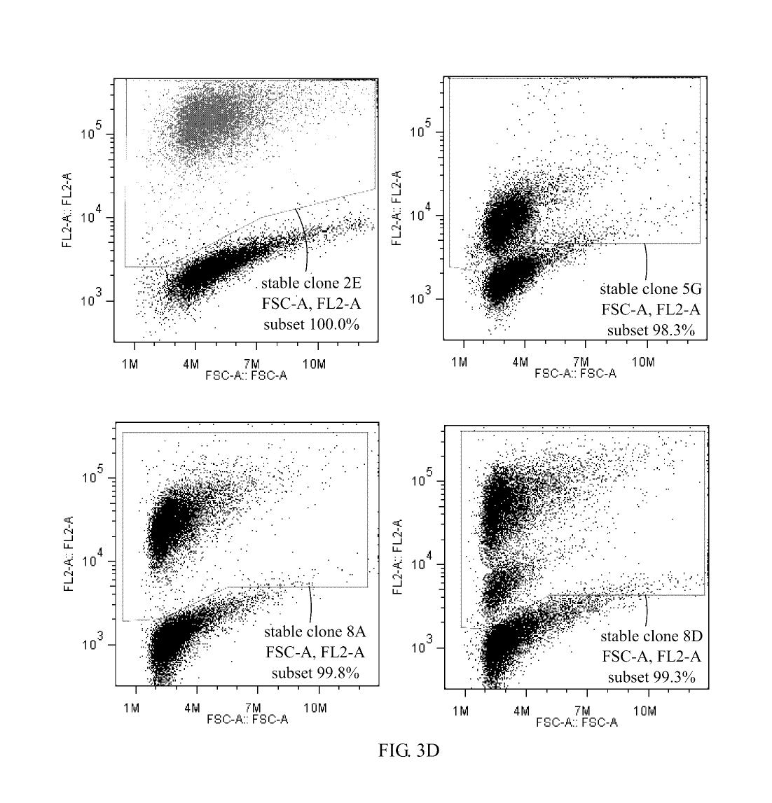

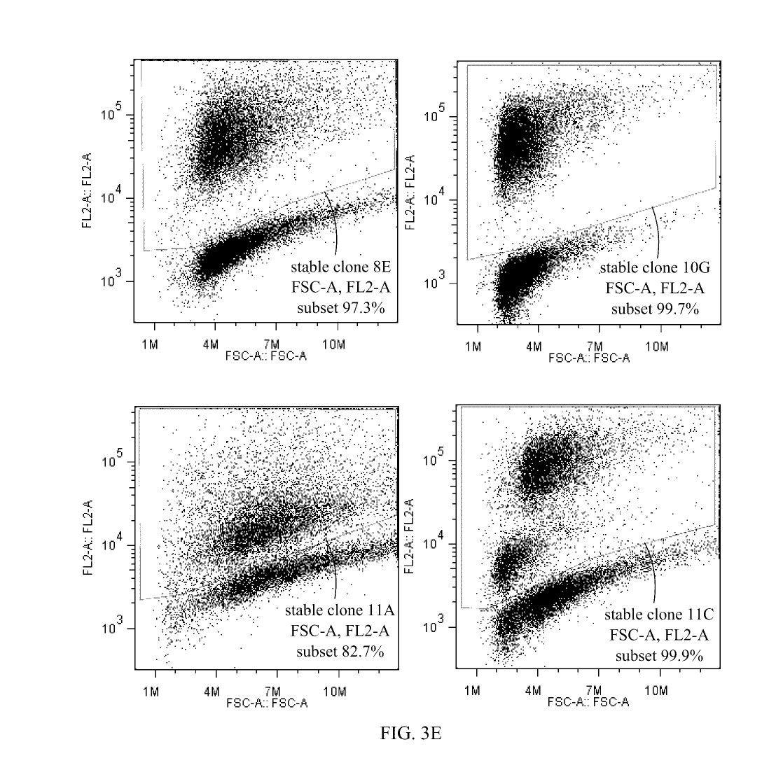

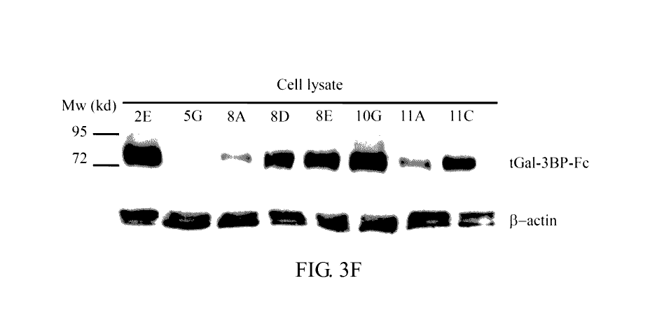

FIG. 3A shows 8 representative stable clones of tGal-3BP-Fc-expressing CHO cells (2E, 5G, 8A, 8D, 8E, 10G, 11A and 11C). In tGal-3BP-Fc-expressing CHO cells, the tGal-3BP-Fc protein in condition medium was detected via anti-human IgAb by using EIA method (FIG. 3B) and Western blot (FIG. 3C). Furthermore, the expression of the tGal-3BP-Fc fusion protein in cytosol was also detected by using flow cytometric assay (FIGS. 3D and 3E, in which the upper portion of each graph showed the result of permeabilizing anti-human IgAb, in comparison to the low portion of each graph showing the result of permeabilizing irrelevant anti-human IgAb) and Western blot (FIG. 3F). Low tGal-3BP-Fc fusion protein expression was seen in clone 5G in both condition medium and cytosol by using Western blot.

Binding of tGal-3BP-Fc Fusion Protein with Gal-1 or Gal-3

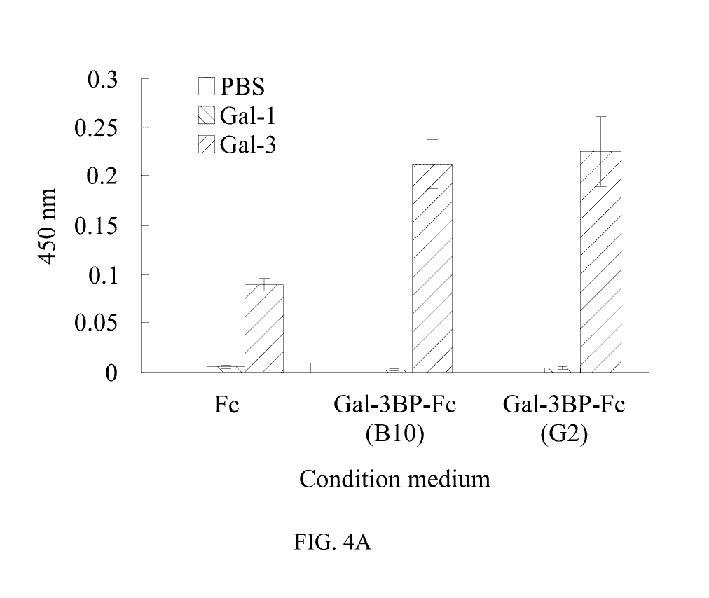

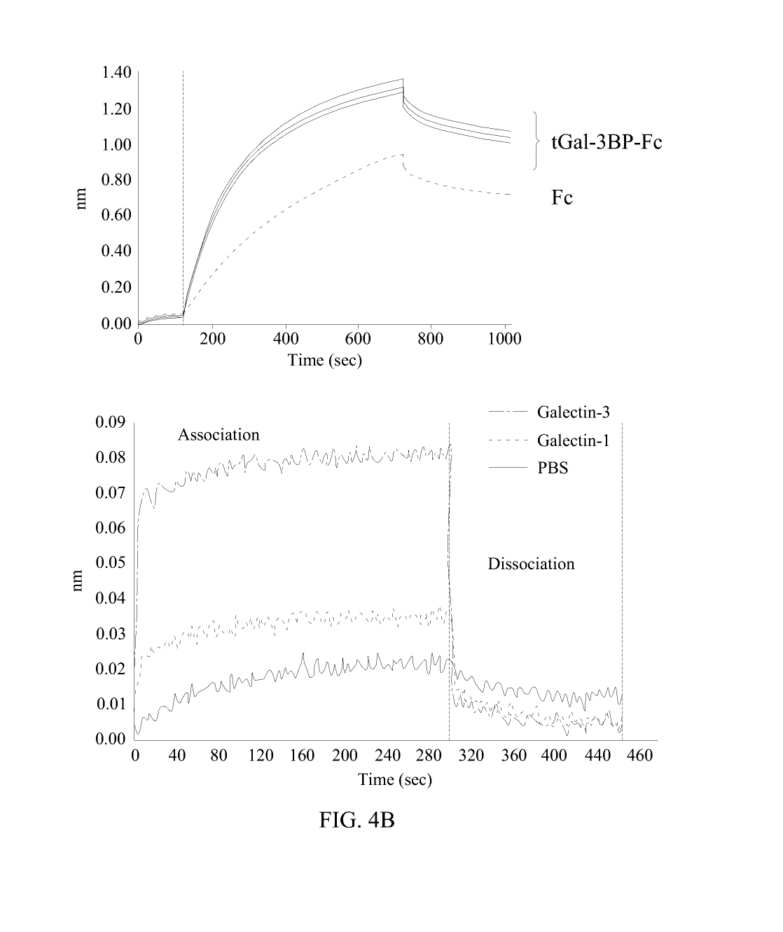

Referring to FIG. 4A, Gal-1 or Gal-3 recombinant protein (250 ng/100 μl) was coated on 96-well plate while PBS was used as a blank control. Condition medium of tGal-3BP-Fc-expressing CHO cells (10B, 2G) was added into Gal-1- or Gal-3-coated wells followed by addition of anti-human Ig-HRP and finally colorimetric detection using TMB. In FIG. 4A, the tGal-3BP-Fc fusion proteins expressed from either clones 10B or 2G specifically bind to Gal-3, instead of binding to Gal-1 or PBS.

Referring to FIG. 4B, tGal-3BP-Fc or Fc protein was immobilized on Protein A Chip (upper). After inactivation of binding sites on chip, Gal-1 or Gal-3 recombinant protein flowed through the chip. Surface plasmon resonance (SPR) was measured by subtracting the binding signals induced by binding of Gal-3 or Gal-1 to Fc-coated chip from the binding signals of Gal-3 or Gal-1 to tGal-3BP-Fc-coated chip. PBS was used as a negative control. In FIG. 4B, Gal-3 shows the strongest binding affinity against the tGal-3BP-Fc-coated chip while Gal-1 shows weak binding affinity against the tGal-3BP-Fc-coated chip.

Binding of tGal-3BP-Fc Fusion Protein and Fc Receptor (FcR)

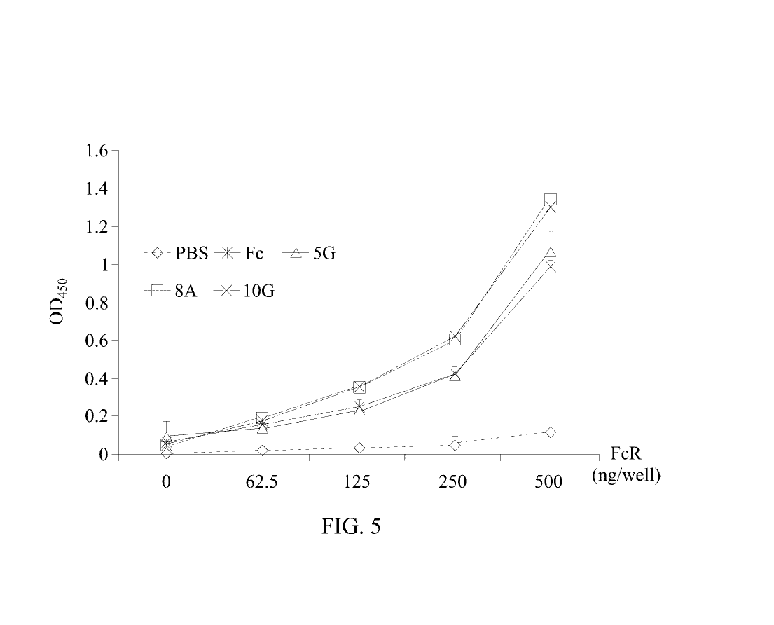

Referring to FIG. 5, FcR recombinant protein (62.5, 125, 250, 500 ng/100 μl) was coated on 96-well plate. PBS was used as a blank control. Condition medium of tGal-3BP-Fc-expressing CHO cells (5G, 8A, 10G) was added into FcR-coated wells followed by addition of anti-human Ig-HRP. Finally, colorimetric detection was performed by using TMB. In FIG. 5, the tGal-3BP-Fc fusion proteins in the condition medium of tGal-3BP-Fc-expressing clones 5G, 8A or 10G successfully bind to the FcR recombinant protein coated on the 96-well plate.

Modulation of T-Cells Apoptosis and Polarization by Depletion of Gal-3 with tGal-3BP-Fc

Referring to FIG. 2A, a schematic representation of the tGal-3BP-Fc modulation of T cell apoptosis and functions. In FIGS. 2B-2C, FcR is coated in wells of a 48-well plate (500 ng/well) for 1 hour and wash the coated well with 1X PBS for 3 times to remove excessive FcR. Pre-incubate tGal-3BP-Fc fusion proteins of various concentrations (0-0.08 μM) with Gal-3 (5 μM) for 1 hour, and add the mixture to the 48-well plate coated with FcR such that the Gal-3 bound to the tGal-3BP-Fc fusion proteins would be fixed in the wells due to Fc-FcR interaction. Remove the mixture containing proteins that were not bound to the FcR from the 48-well plate, and analyze the mixture by Western blot (FIG. 2B), in which anti-Fc antibody and anti-Gal-3 antibody were applied to verify the amount of the tGal-3BP-Fc fusion proteins and Gal-3 in the mixture. Furthermore, Jurkat T cells are incubated with the mixture removed from the 48-well plate, and the cells are further analyzed for their survival by Annix-PI stain assay using flow cytometry.

As shown in FIG. 2B, the amount of the Gal-3 in the mixture removed from the 48-well plate gradually decreases as the concentration of the tGal-3BP-Fc fusion proteins in the original mixture increases. That is, the Gal-3 in free form in the mixture removed from the 48-well plate decreases as the tGal-3BP-Fc fusion proteins in the original mixture increases. Referring to FIG. 2C, the presence of Gal-3 in free form leads to apoptosis of the Jurkat T cells. However, as the concentration of the tGal-3BP-Fc fusion proteins increase, indicating the Gal-3 in free form decrease, the apoptosis of the Jurkat T cells is alleviated. In other words, the tGal-3BP-Fc fusion proteins show an inhibitory effect on T-cell apoptosis by reducing the Gal-3 in free form. Referring to FIG. 2D, Jurkat T cells are incubated with the mixture removed from the 48-well plate, and the cells are further analyzed for their transcription factor T-bet expression of T help cells polarization.

Phagocytosis of tGal-3BP-Fc Fusion Protein in PMA-Treated THP-1 Macrophages

Referring to FIG. 6, after incubation of tGal-3BP-Fc fusion protein with PMA-treated THP-1 macrophages, FITC-conjugated anti-human IgAb was used to detect cytosolic tGal-3BP-Fc fusion protein phagocytosed in permeabilized THP-1 macrophages with fluorescence microscope. DAPI was used for staining nuclei (blue nuclei). In FIG. 6, small aggregation of fluorescent signals was found in tGal-3BP-Fc-incubated THP-1 macrophages. That is, the tGal-3BP-Fc fusion protein has been engulf into the THP-1 macrophages.

Inhibitory Effects of tGal-3BP-Fc on Binding of Gal-3 to mIgE

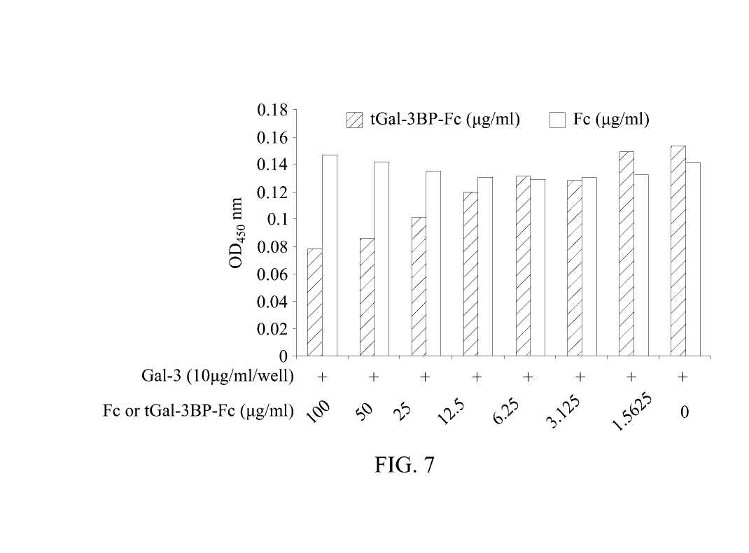

Referring to FIG. 7, mIgE was coated on 96-well plate and Gal-3 (10 μg/ml) showed the binding affinity to mIgE. Pre-incubation of biotin-conjugated Gal-3 and tGal-3BP-Fc or Fc on ice for 30 min was followed by adding into mIgE-coated wells. Colorimetric detection was performed by using streptavidin conjugated HRP and TMB substrate. The tGal-3BP-Fc fusion protein showed inhibiting effects on binding of Gal-3 and mIgE in a dose-dependent manner from 12.5 to 100 μg/ml.

Gal-3 Induced Migration of Gal-3-Knockdown DLD-1 cells (DLD-1 shGal-3) and Inhibited by Addition of tGal-3BP-Fc Fusion Protein

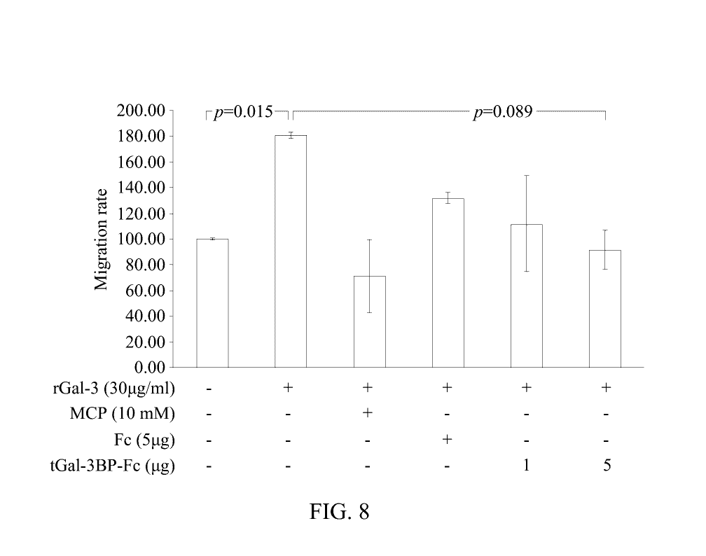

Referring to FIG. 8, endogenous Gal-3 in DLD-1 cells was knockdown and recombinant Gal-3 was transfected into the Gal-3-knockdown DLD-1 cells. Modified Citrus Pectin (MCP) was used as a positive control for inhibiting Gal-3 effects. In FIG. 8, migration was enhanced by introducing recombinant Gal-3 into the Gal-3-knockdown DLD-1 cells. However, the Gal-3-induced migration of Gal-3-knockdown DLD-1 cells was inhibited by either MCP or tGal-3BP-Fc fusion protein (1, 5 μg). That is, the tGal-3BP-Fc fusion protein obviously inhibits, decreases, reduces, suppresses or limits migration and metastasis of cancer cells.

### CONCLUSIONS

In summary, tGal-3BP-Fc fusion protein was successfully constructed. Also, the purified tGal-3BP-Fc fusion protein is well functioned, demonstrated by the binding of tGal-3BP-Fc fusion protein with Gal-3 and Fc receptor. In addition, the tGal-3BP-Fc fusion protein may further phagocytosed by THP-1 macrophages, indicated that the tGal-3BP-Fc fusion protein may trigger cellular immune response, thereby potentially killing the targeted Gal-3-overexpressed cancer cells. On the other hand, tGal-3BP-Fc fusion protein may inhibit, decrease, reduce, suppress or limit migration, even metastasis of cancer cells, and modulate macrophage phagocytosis and T-cell functions. Accordingly, tGal-3BP-Fc fusion proteins would be an excellent candidate of specific target therapies for simultaneously suppression of cancer invasiveness and enhancement of anti-tumor immunity, and target therapies for immunosuppression or modulation of inflammatory disorders such as autoimmune diseases.

While the means of specific embodiments in present invention has been described by reference drawings, numerous modifications and variations could be made thereto by those skilled in the art without departing from the scope and spirit of the invention set forth in the claims. The modifications and variations should be in the range limited by the specification of the present invention.

What is claimed is:

1. A tGal-3BP-Fc fusion protein, comprising:
a truncated Gal-3 binding protein with domain 4; and
at least an Fc fragment of an immunoglobulin G.

2. The fusion protein of claim 1, wherein the truncated Gal-3 binding protein further comprises a fragment having at least a glycosylation side of domain 3 of Gal-3 binding protein.

3. The fusion protein of claim 1, wherein the Fc fragment is linked to a C-terminus or N-terminus of the truncated Gal-3 binding protein.

4. The fusion protein of claim 3, wherein the Fc fragment comprises binding affinities against Fc receptors on white blood cells.

5. The fusion protein of claim 1, wherein the truncated Gal-3 binding protein comprises N-glycosylation for binding of Gal-1, Gal-3 and/or other galectins.

6. The fusion protein of claim 1, wherein the truncated Gal-3 binding protein comprises of a sequence of SEQ ID NO: 2.

7. The fusion protein of claim 2, wherein the truncated Gal-3 binding protein consists of a sequence of SEQ ID NO: 4.

8. The fusion protein of claim 1, wherein the immunoglobulin comprises human IgG1 or IgG3.

9. A recombinant expression vector comprising the isolated nucleic acid encoding the fusion protein of claim 1.

10. A cell transformed or transfected with the recombinant expression vector of claim 9.

11. The cell of claim 10, comprising mammalian cells.

12. A pharmaceutical composition for inhibiting, decreasing, reducing, suppressing or limiting metastasis of cancer cells, comprising:
the fusion protein of claim 1; and
a pharmaceutically acceptable carrier for inhibiting, decreasing, reducing, suppressing or limiting invasiveness and metastasis of cancer cells.

13. The pharmaceutical composition of claim 12, wherein the cancer cells overexpresses galectin-1, galectin-3 or a combination thereof on the cell surface, in extracellular spaces or in blood circulation.

14. The pharmaceutical composition of claim 13, wherein the cancer cells is selected from a group consisting of colon cancer, rectal cancer, breast cancer, cervical cancer, gastric cancer, pancreatic cancer, prostate cancer, renal cancer, melanoma, and ovarian cancer.

15. The pharmaceutical composition of claim 12, wherein the pharmaceutical composition is administered orally, intramuscularly, intraperitoneally, intravenously, inhaled, or subcutaneously.

16. A method of modulating immune functions, comprising:
administering the pharmaceutical composition comprising the fusion protein of claim 1 and a pharmaceutically acceptable carrier to a subject in a therapeutically effective amount, to compete with galectin-3 and galectin-3 binding protein (90K), and resulting in modulation of proinflammatory reactions or adaptive T cell polarization.

17. The method of claim 16, wherein the subject is a mammal.

18. The method of claim 16, wherein the proinflammatory reactions or the adaptive T cell polarization is related to a disease comprising rheumatoid arthritis, autoimmune diseases, inflammatory diseases or macrophage activation syndromes.

19. The method of claim 16, wherein the pharmaceutical composition is administered orally, intramuscularly, intraperitoneally, intravenously, inhaled, or subcutaneously.
